(19) United States
(12) Patent Application Publication
(10) Pub. No.: US 2009/0171413 A1
(43) Pub. Date: Jul. 2, 2009

(54) IMPLANTABLE DEVICE, SYSTEM INCLUDING SAME, AND METHOD UTILIZING SAME

(76) Inventors: Marco Zenati, Pittsburgh, PA (US); William W. Clark, Wexford, PA (US); Robert J. Sclabassi, Gibsonia, PA (US); Mingui Sun, Pittsburgh, PA (US); Sung Kwon Cho, Pittsburgh, PA (US); Hsin-Hua Hu, Chubei (TW)

Correspondence Address: REED SMITH LLP, P.O. BOX 488, PITTSBURGH, PA, 15230-0488, US

(21) Appl. No.: 12/203,041

(22) Filed: Sep. 2, 2008

Related U.S. Application Data
(60) Provisional application No. 60/969,415, filed on Aug. 31, 2007.

(51) Int. Cl. A61N 1/362; A61B 5/145

(52) U.S. Cl. 607/32; 600/309; 607/35

(57) ABSTRACT

An implantable device. The implantable device includes a computing device, a microelectromechanical system (MEMS) pH sensor connected to the computing device, and a communication system connected to the computing device.

### CROSS-REFERENCE TO RELATED APPLICATION

This application claims the benefit of the earlier filing date of U.S. Patent Provisional Application No. 60/969,415 filed on Aug. 31, 2007.

### BACKGROUND

This application discloses an invention which is related, generally and in various embodiments, to an implantable device, a system including the implantable device, and a method utilizing the implantable device.

Under a variety of circumstances, human organs (e.g., heart, brain, liver, kidney, lung, etc.) can become at risk for ischemia. For example, acute coronary syndromes include a spectrum of conditions associated with acute myocardial ischemia. These conditions are a major cause of morbidity and mortality around the world. Often, the signs and symptoms related to acute coronary syndromes occur without warning. One such symptom, angina pectoris, occurs when an area of the heart does not receive enough oxygen-rich blood. For patients with angina pectoris, the patients commonly mistake the symptoms for gastric acid reflux, indigestion, arthritic pain, etc. In other instances, the signs and symptoms related to acute coronary syndromes are not even perceived by the person—the signs and symptoms are “silent”.

Unfortunately, the mistaken diagnosis or the lack of apparent symptoms often delays referral to a hospital emergency department for prompt treatment. Without timely and aggressive pharmacological and device-based therapy, acute coronary syndromes often evolve into myocardial infarction, eventually leading to serious complications including myocardial cell death, ventricular arrhythmias, heart failure, and death. Similarly, other types of organ ischemia also often lead to serious complications.

It is generally accepted that patients treated in the first hour following the onset of myocardial ischemia have the highest absolute and relative mortality benefit. Thus, it is beneficial to detect impending acute coronary syndromes, and to provide suitable treatment prior to the occurrences of the symptoms. Similarly, it is beneficial to detect other types of impending organ ischemia and provide suitable treatment as early as possible.

For a patient who experiences acute coronary syndromes, makes it to the hospital, and survives, a device may be surgically implanted to monitor pressures within the circulatory system (e.g., within an abdominal aortic aneurysm sac). Although such monitoring provides a certain peace of mind, the device is less than optimal because it does not predict the occurrence of subsequent acute coronary syndromes, and does not provide any treatment of such subsequent acute coronary syndromes.

### SUMMARY

In one general respect, this application discloses an implantable device. According to various embodiments, the implantable device includes a computing device, a microelectromechanical system (MEMS) pH sensor connected to the computing device, and a communication system connected to the computing device.

In another general respect, this application discloses a system. According to various embodiments, the system includes an implantable device, and a communication device connected to the implantable device. The implantable device includes a computing device, a microelectromechanical system (MEMS) pH sensor connected to the computing device, and a communication system connected to the computing device.

In yet another general respect, this application discloses a method, implemented at least in part by a computing device. According to various embodiments, the method includes measuring pH values of an organ with an implanted device, and determining whether organ ischemia exists based on at least one of the measured pH values.

Aspects of the invention may be implemented by a computing device and/or a computer program stored on a computer-readable medium. The computer-readable medium may comprise a disk, a device, and/or a propagated signal.

### BRIEF DESCRIPTION OF THE DRAWINGS

Various embodiments of the invention are described herein in by way of example in conjunction with the following figures, wherein like reference characters designate the same or similar elements.

FIG. 1 illustrates various embodiments of an implantable device;

FIG. 2 illustrates various embodiments of a computing device of the implantable device of FIG. 1;

FIG. 3 illustrates various embodiments of a MEMS pH sensor of the implantable device of FIG. 1;

FIG. 4 illustrates various embodiments of a MEMS pH sensor of the implantable device of FIG. 1;

FIG. 5 illustrates various embodiments of a MEMS pressure sensor of the implantable device of FIG. 1;

FIG. 6 illustrates various embodiments of a communication system of the implantable device of FIG. 1;

FIG. 7 illustrates various embodiments of a volume conduction antenna of the communication system of FIG. 5;

FIG. 8 illustrates various embodiments of a communication system of the implantable device of FIG. 1;

FIG. 9 illustrates various embodiments of a system which includes the implantable device of FIG. 1;

FIG. 10 illustrates various embodiments of a communication device of the system of FIG. 9;

FIG. 11 illustrates various embodiments of a power source of the system of FIG. 9;

FIG. 12 illustrates various embodiments of a power source of the system of FIG. 9;

FIG. 13 illustrates various embodiments of a power source of the system of FIG. 9;

FIG. 14 illustrates various embodiments of a power source of the system of FIG. 9; and

FIG. 15 illustrates various embodiments of a method which utilizes the implantable device of FIG. 1.

### DETAILED DESCRIPTION

It is to be understood that at least some of the figures and descriptions of the invention have been simplified to illustrate elements that are relevant for a clear understanding of the invention, while eliminating, for purposes of clarity, other elements that those of ordinary skill in the art will appreciate may also comprise a portion of the invention. However, because such elements are well known in the art, and because they do not facilitate a better understanding of the invention, a description of such elements is not provided herein.

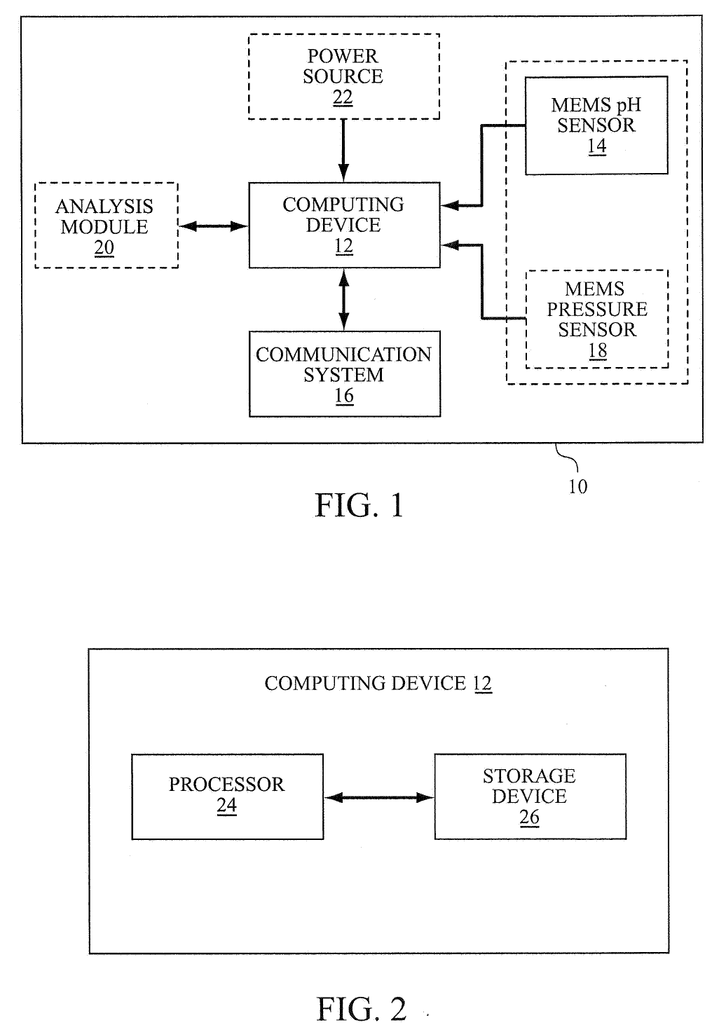

FIG. 1 illustrates various embodiments of an implantable device 10. The implantable device 10 is of a size and configuration which is suitable for implantation on an organ (e.g., heart, brain, liver, kidney, lung, etc.), and may be implanted using a minimally invasive technique. The implantable device 10 may be utilized for the detection and treatment of organ ischemia. The implantable device 10 includes a computing device 12, a microelectromechanical system (MEMS) pH sensor 14, and a communication system 16. As shown in FIG. 1, according to various embodiments, the implantable device 10 may also include a MEMS pressure sensor 18, an analysis module 20, and a power source 22.

The computing device 12 may be any suitable type of computing device. For example, according to various embodiments, the computing device 12 is configured as shown in FIG. 2. For such embodiments, the computing device 12 includes a processor 24. The processor 24 may be any suitable type of processor (e.g., a microprocessor, a digital signal processor, etc.). As shown in FIG. 2, according to various embodiments, the computing device 12 also includes a storage device 26. The storage device 26 may be any suitable type of storage device. According to various embodiments, the computing device 12 is configured for direct memory access.

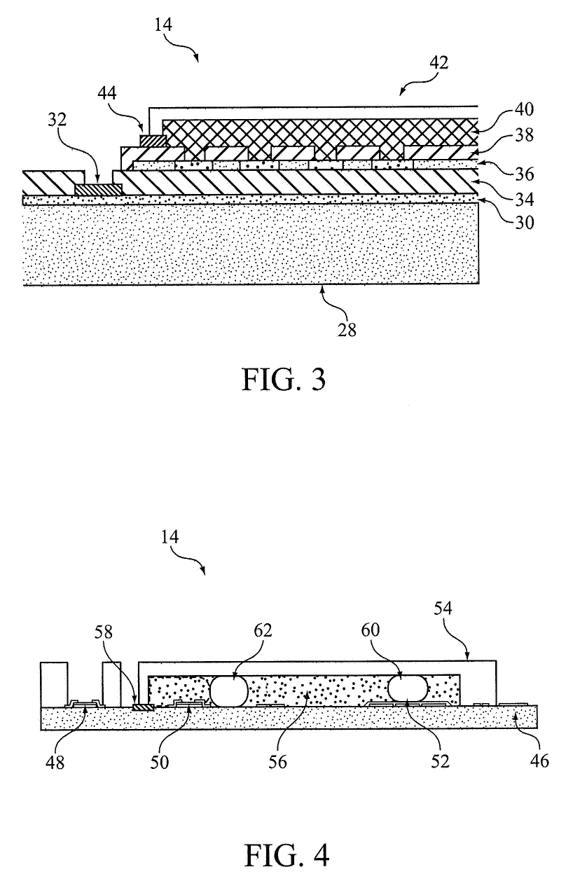

The MEMS pH sensor 14 is connected to the computing device 12, and is configured for continuously measuring a pH level (e.g., a pH level of an organ). The MEMS pH sensor 14 may be any suitable type of MEMS pH sensor. For example, according to various embodiments, the MEMS pH sensor 14 is configured as shown in FIG. 3. For such embodiments, the MEMS pH sensor 14 includes a substrate 28, a first electrode 30, a second electrode 32, a first dielectric layer 34, a third electrode 36, a second dielectric layer 38, an electrolyte layer 40, a passivation layer 42, and a liquid junction 44. The liquid junction 44 provides an electrical connection between the electrolyte layer 40 and tissue fluid of the organ of which pH is to be measured (e.g., myocardial tissue fluid, brain tissue fluid, liver tissue fluid, kidney tissue fluid, lung tissue fluid, etc.).

The first electrode 30 functions as an internal reference electrode, and may include any suitable type of conductor (e.g., gold). The second electrode 32 functions as an indicator electrode, and may include any suitable type of conductor (e.g., iridium oxide). The third electrode 36 functions as a reference electrode, and may include any suitable type of conductor (e.g., silver, silver chloride).

According to other embodiments, the MEMS pH sensor 14 is configured as shown in FIG. 4. For such embodiments, the MEMS pH sensor 14 includes a substrate 46, a first electrode 48, a second electrode 50, a plurality of third electrodes 52, a cover 54, a fluidic channel 56, and a liquid junction 58. The plurality of third electrodes 52 and the fluidic channel 56 cooperate to form a microfluidic switch.

The first electrode 48 functions as an indicating electrode, and may include any suitable type of conductor (e.g., platinum, chromium, titanium, iridium oxide). The second electrode 50 functions as a reference electrode, and may include any suitable type of conductor (e.g., platinum, chromium, titanium, silver, silver chloride). The plurality of third electrodes 52 collectively function as a microfluidic switch, and the microfluidic switch may include any suitable type of conductor (e.g., platinum, chromium, titanium, etc.), any suitable type of insulating layer (e.g., silicon oxide, parylene, etc.), and any suitable type of hydrophobic layer (e.g., a fluorocarbon hydrophobic layer). The fluidic channel 56 includes a first bubble 60 and a second bubble 62. Each of the first and second bubbles 60, 62 are movable, and are hydrodynamically connected to one another.

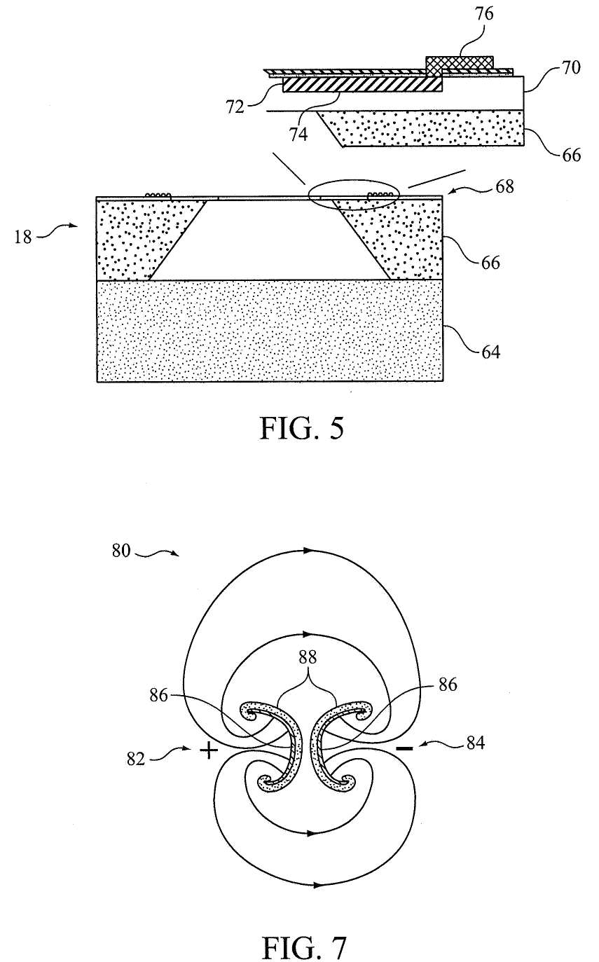

The MEMS pressure sensor 18 is connected to the computing device 12, and is configured for continuously measuring a tension level (e.g., a left ventricular wall tension level). The MEMS pressure sensor 18 may be any suitable type of MEMS pressure sensor. For example, according to various embodiments, the MEMS pressure sensor 18 is configured as shown in FIG. 5. For such embodiments, the MEMS pressure sensor 18 includes a base 64, a substrate 66, and a pressure sensing membrane 68. As shown in the exploded portion of FIG. 5, according to various embodiments, the membrane 68 includes a base layer 70, a piezoresistive sensing member 72, a wire lead 74, and a metal layer 76. As shown conceptually in FIG. 1, the MEMS pH sensor 14 and the MEMS pressure sensor 18 may be incorporated into a single MEMS device.

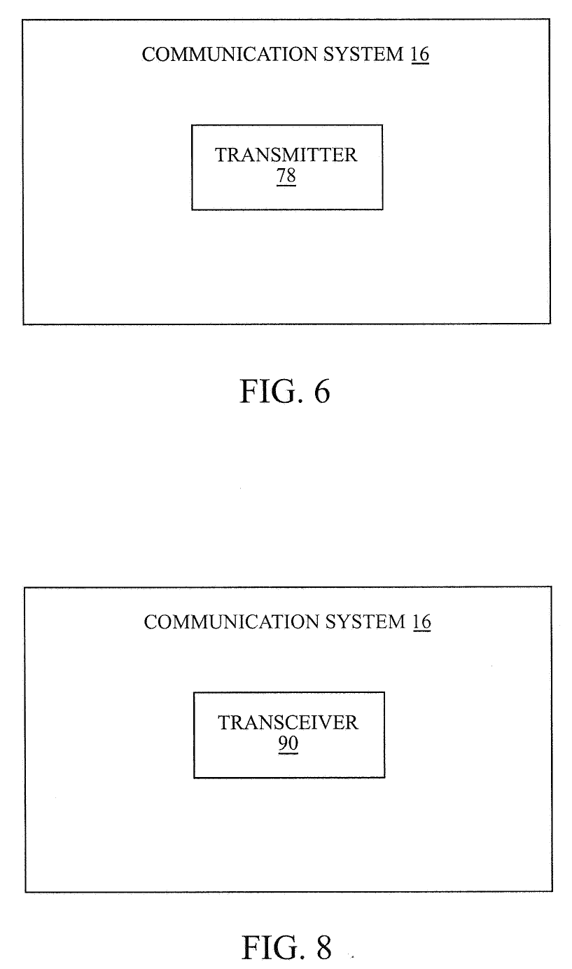

The communication system 16 is connected to the computing device 12, and is configured for sending information from the implantable device 10. The communication system 16 may be any suitable type of communication system. For example, according to various embodiments, the communication system 16 is configured as shown in FIG. 6. For such embodiments, the communication system 16 includes a transmitter 78 connected to the computing device 12.

The transmitter 78 may be any suitable type of transmitter. For example, according to various embodiments, the transmitter 78 is a radio-frequency transmitter. According to other embodiments, the transmitter 78 is a volume conduction transmitter. For embodiments where the transmitter 78 is a volume conduction transmitter, the transmitter 78 includes a volume conduction antenna 80 (see FIG. 7). The volume conduction antenna 80 may be any suitable type of volume conduction antenna, and may have any suitable shape. For example, according to various embodiments, the volume conduction antenna 80 may be configured as shown in FIG. 7. For such embodiments, the volume conduction antenna 80 is a dipole antenna which includes a first pole 82 and a second pole 84. Each of the first and second poles 82, 84 includes a conductive layer 86, and an insulating layer 88 connected to the conducting layer 86. As the shorting paths between the two poles 82, 84 are blocked by the respective insulating layers 88, current is forced to flow along much longer paths, thereby significantly enhancing the far-field which contributes to the transmission of information from the volume conduction antenna 80.

According to various embodiments, the communication system 16 is also configured for receiving information sent to the implantable device 10. For such embodiments, the communication system 16 either includes a receiver (not shown) in addition to the transmitter 78, or a transceiver 90 in lieu of the transmitter 78 as shown in FIG. 8.

The analysis module 20 is configured for determining the existence of organ ischemia based at least in part on one or more of the pH values of the organ (e.g., heart, brain, liver, kidney, lung, etc.) measured by the MEMS pH sensor 14. According to various embodiments, the analysis module 20 is further configured for determining the existence of organ ischemia based at least in part on one or more of the measured organ pH values and one or more of the left ventricular wall tension values measured by the MEMS pressure sensor 18. The analysis module 20 may be implemented in hardware, firmware, software and combinations thereof. For embodiments utilizing software, the software may utilize any suitable computer language (e.g., C, C++, Java, JavaScript, Visual Basic, VBScript, Delphi) and may be embodied permanently or temporarily in any type of machine, component, physical or virtual equipment, storage medium, or propagated signal capable of delivering instructions to a device. The analysis module 20 (e.g., software application, computer program) may be stored on a computer-readable medium (e.g., disk, device, and/or propagated signal) such that when a computer reads the medium, the functions described herein are performed.

According to various embodiments, the analysis module 20 may reside at the computing device 12, at another component of the implantable device 10, or combinations thereof. For embodiments where the implantable device 10 includes more than one computing device 12, the analysis module 20 may be distributed across two or more computing devices 12.

The power source 22 is configured to provide power to the components of the implantable device 10, and is connected to the computing device 12. The power source 22 may be any suitable type of power source. For example, according to various embodiments, the power source 22 may be a rechargeable battery, a non-rechargeable battery, etc.

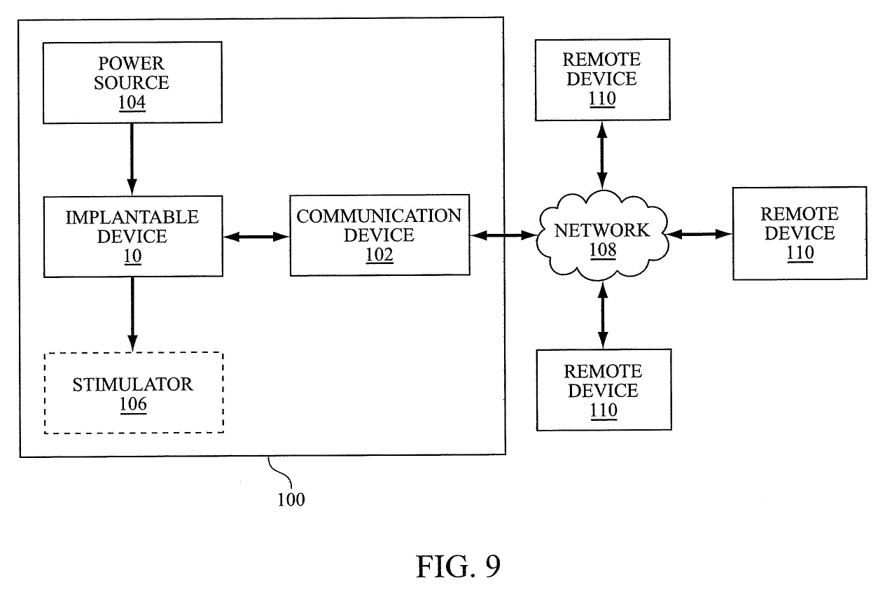

FIG. 9 illustrates various embodiments of a system 100. The system 100 may be utilized for the detection of organ ischemia. For example, the system 100 may be utilized to detect ischemia of a heart, a brain, a liver, a kidney, a lung, etc. According to various embodiments, the system 100 may also be utilized for the treatment of organ ischemia (e.g., treatment of myocardial ischemia). The system 100 includes the implantable device 10 of FIG. 1, and also includes a communication device 102 communicably connected to the implantable device 10. The communication device 102 is positioned external to the body, and may be communicably connected to the implantable device 10 in any suitable manner. For example, the communication device 102 may be wirelessly connected to implantable device 10 via volume conduction, via radio frequency inductive coupling, etc. As shown in FIG. 9, according to various embodiments, the system 100 may also include a power source 104 connected to the implantable device 10, and a stimulator 106 connected to either the implantable device 10 or the communication device 102.

As shown in FIG. 9, the communication device 102 may also be communicably connected to a network 108 having wired or wireless data pathways, and may also be communicably connected to a plurality of remote devices 110 (e.g., a device associated with emergency medical personnel) via the network 108. The network 108 may include any type of delivery system including, but not limited to, a local area network (e.g., Ethernet), a wide area network (e.g. the Internet and/or World Wide Web), a telephone network (e.g., analog, digital, wired, wireless, PSTN, ISDN, GSM, GPRS, and/or xDSL), a packet-switched network, a radio network, a television network, a cable network, a satellite network, and/or any other wired or wireless communications network configured to carry data. The network 108 may include elements, such as, for example, intermediate nodes, proxy servers, routers, switches, and adapters configured to direct and/or deliver data. In general, the communication device 102 is configured to communicate with the remote devices 110 via the network 108 using various communication protocols (e.g., HTTP, TCP/IP, UDP, WAP, WiFi, Bluetooth) and/or to operate within or in concert with one or more other communications systems.

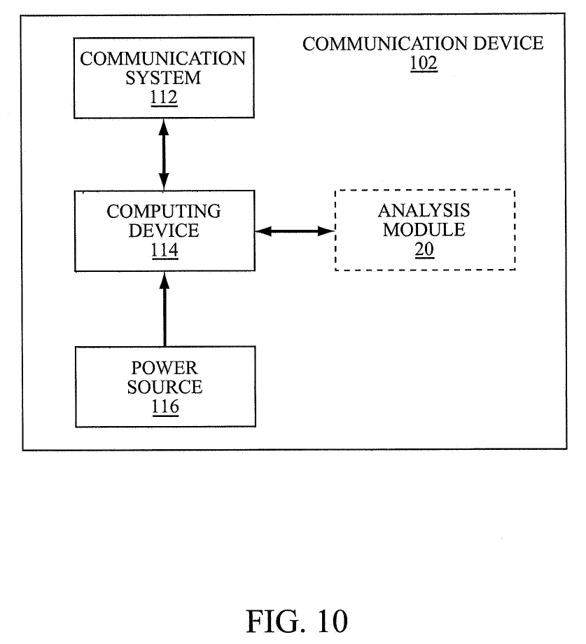

The communication device 102 is configured for receiving information sent from the implantable device 10. According to various embodiments, the communication device 102 is also configured for sending information to the implantable device 10. The communication device 102 may be any suitable type of communication device. For example, according to various embodiments, the communication device 102 is configured as shown in FIG. 10. For such embodiments, the communication device 102 includes a communication system 112, a computing device 114, and a power source 116. As shown in FIG. 10, according to various embodiments, the communication device 102 may also include the analysis module 20 (or portions thereof).

The communication system 112 may be any suitable type of communication system. For example, according to various embodiments, the communication system 112 is configured similar to the communication system 16. The computing device 114 may be any suitable type of computing device. For example, according to various embodiments, the computing device 114 is configured similar to the computing device 12. The power source 116 may be any suitable type of power source. For example, according to various embodiments, the power source 116 is configured similar to the power source 22.

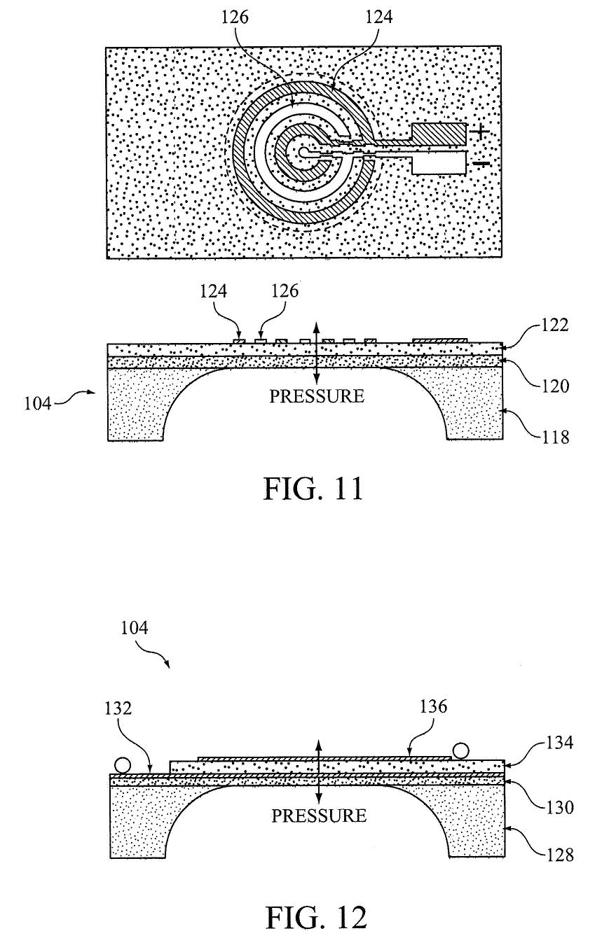

The power source 104 of the system 100 is configured to provide power to the components of the implantable device 10. The power source 104 may be any suitable type of power source. For example, according to various embodiments, the power source 104 is a piezoelectric energy harvesting device configured for converting one or more body forces into electricity. The piezoelectric energy harvesting device may be any suitable type of piezoelectric energy harvesting device. For example, according to various embodiments, the piezoelectric energy harvesting device 104 may be configured as shown in FIG. 11 or as shown in FIG. 12.

The piezoelectric energy harvesting device 104 of FIG. 11 includes a base 118, a carrying layer 120, a piezoelectric material 122, a first electrode 124, and a second electrode 126. As shown in the top view portion of FIG. 11, the first and second electrodes 124, 126 are interdigitated. The piezoelectric energy harvesting device 104 of FIG. 12 includes a base 128, a carrying layer 130, a first electrode 132, a piezoelectric material 134, and a second electrode 136.

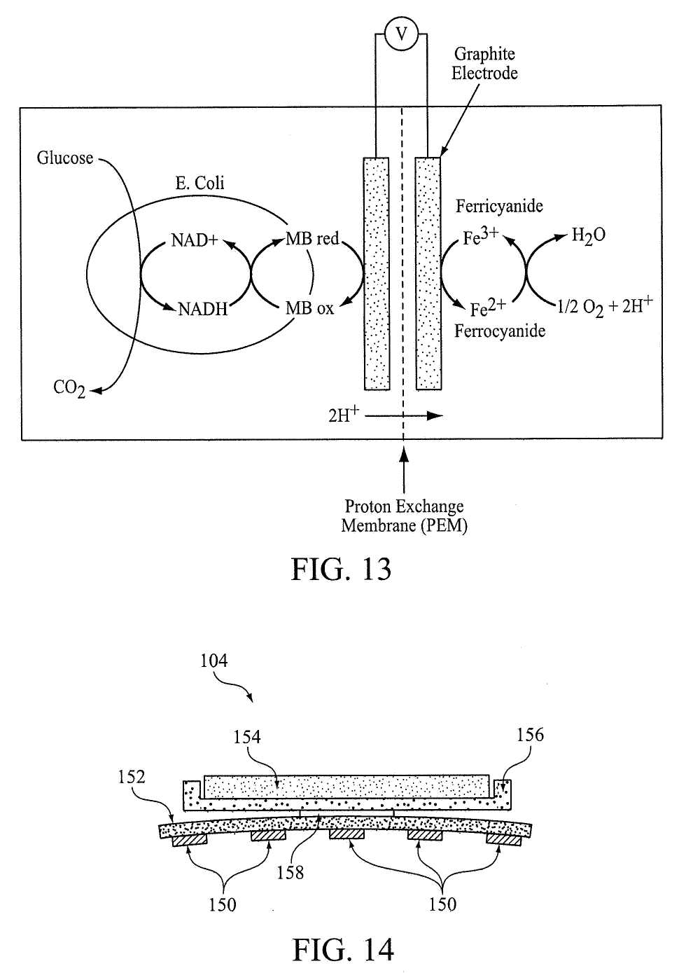

According to other embodiments, the power source 104 is a biofuel cell. The biofuel cell may be any suitable type of biofuel cell. For example, according to various embodiments, the biofuel cell 104 may be configured as shown in FIG. 13. For such embodiments, the biofuel cell 104 couples the oxidation of a biofuel (e.g., glucose) to the reduction of molecular oxygen to water and outputs electricity.

According to other embodiments, the power source 104 is a volume conduction energy delivery device. The volume conduction energy delivery device may be any suitable type of volume conduction energy delivery device. For example, according to various embodiments, the volume conduction energy delivery device 104 may be configured as shown in FIG. 14. For such embodiments, the volume conduction energy delivery device 104 includes a plurality of electrodes 150, a disposable pad 152, a power source 154 (e.g., a battery), a printed circuit board 156, and a connector 158.

The stimulator 106 is an implantable stimulator which is connected to the implantable device 10 and to a part of the body (e.g., a cardiac vagal nerve branch). The stimulator 106 is configured to deliver a current to the part of the body when the implantable device 10 applies a voltage across the stimulator 106. The stimulator 106 may be any suitable type of stimulator.

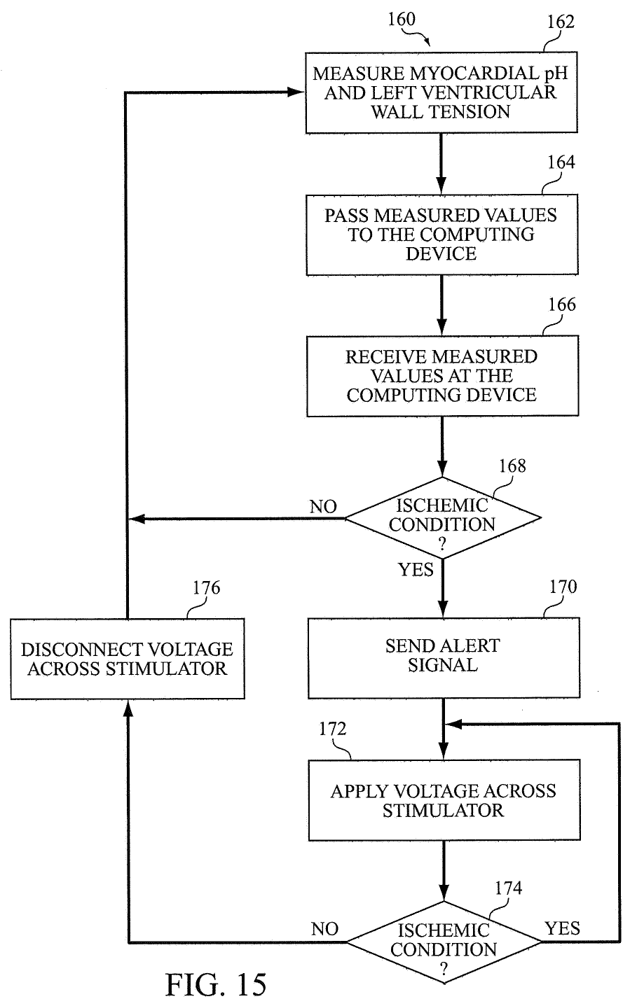

FIG. 15 illustrates various embodiments of a method 160. The method 160 is implemented at least in part by a computing device, and may be implemented by the system 100 of FIG. 9. The method 160 may be utilized, for the detection of organ ischemia. For example, the method 160 may be utilized to detect ischemia of a heart, a brain, a liver, a kidney, a lung, etc. According to various embodiments, the method 160 may also be utilized for the treatment of organ ischemia (e.g., treatment of myocardial ischemia). For ease of description purposes, the method 160 will be described in the context of its implementation by the system 100 of FIG. 9 for the detection and treatment of myocardial ischemia. However, it will be appreciated that the method 160 may be implemented by other systems and may be utilized for the detection and treatment of other types of organ ischemia.

Prior to the start of the process, the implantable device 10 is implanted into a body in a manner which allows the MEMS pH sensor 14 to measure the myocardial pH. According to various embodiments, the implantation of the implantable device 10 also allows the MEMS pressure sensor 18 to measure the left ventricular wall tension of the heart. The stimulator 106 is implanted into the body in a manner which allows for its connection to the implantable device 10 and to one or more cardiac vagal nerve branches.

The process starts at block 162, where the MEMS pH sensor 14 and the MEMS pressure sensor 18 respectively measure the myocardial pH level and the left ventricular wall tension of the heart. The process at block 162 may be repeated any number of times on an on going basis, resulting in the MEMS pH sensor 14 and the MEMS pressure sensor 18 respectively measuring a sequence of myocardial pH levels and a sequence of left ventricular wall tensions.

From block 162, the process advances to block 164, where the respective measured values are passed on to the computing device 12. Due to the electrical connection between the MEMS pH sensor 14 and the computing device 12, the measured myocardial pH values are passed on to the computing device 12 in real time. Similarly, due to the electrical connection between the MEMS pressure sensor 18 and the computing device 12, the measured left ventricular wall tension values are passed on to the computing device 12 in real time.

From block 164, the process advances to block 166, where the computing device 12 receives the measured myocardial pH values and the measured left ventricular wall tension values. From block 166, the process advances to block 168, where the analysis module 20 determines whether a myocardial ischemic condition exists based on one or more of the received myocardial pH values. As described hereinabove, the analysis module 20 may also make the determination based on a combination of one or more of the measured myocardial pH values and one or more of the received left ventricular wall tension values. The analysis module 20 may make this determination any number of times on an on going basis.

The analysis module 20 may make this determination in any suitable manner. For example, according to various embodiments, the analysis module 20 may determine the existence of myocardial ischemia when the measured myocardial pH level drops below a certain threshold value (e.g., 7.3), when the measured myocardial pH level is decreasing at a rate which exceeds a certain threshold rate, etc. According to other embodiments, the analysis module 20 may determine the existence of myocardial ischemia when the measured myocardial pH level drops below a certain threshold value and the measured left ventricular wall tension drops below a certain threshold value, when some combination of measured myocardial pH value and measured left ventricular wall tension value falls within a certain predetermined range, when the measured myocardial pH level is decreasing at a rate which exceeds a certain threshold rate and the measured left ventricular wall tension value is increasing at a rate which exceeds a certain threshold rate, etc.

According to various embodiments, prior to the determination by the analysis module 20, the measured myocardial pH values and if applicable, the measured left ventricular wall tension values, are stored at the storage device 26. For such embodiments, the analysis module 20 accesses the stored values, either directly or via the processor 24, to make the determination as to whether or not the values indicate the existence of organ ischemia. According to other embodiments, the analysis module 20 makes the determination as the measured values are received by the computing unit.

From block 168, the process returns to block 162 or advances to block 170. If the determination made at block 168 is a determination that the measured myocardial pH values and/or the measured left ventricular wall tension values are not indicative of myocardial ischemia, the process returns to block 162, where the process advances as described above. The process described for blocks 162-168 may be repeated any number of times.

If the determination made at block 168 is a determination that the measured myocardial pH values and/or the measured left ventricular wall tension values are indicative of myocardial ischemia, the process advances from block 168 to block 170. At block 170, the implantable device 10 sends a signal (e.g., an alert signal) to the communication device 102, which may in turn send a signal (e.g., an alert signal) to one or more remote devices 110 to alert the appropriate personnel of the organ ischemia. From block 170, the process advances to block 172, where a voltage is applied across the stimulator 106. The voltage may be applied for any period of time, and may be applied as a series of pulses at a predetermined frequency. The application of the voltage stimulates the cardiac vagal nerve branches, which in turn increases the parasympathetic tone. The increase in the parasympathetic tone operates to reduce the myocardial oxygen consumption, which in turn allows for the re-establishment of myocardial biochemical homeostasis. For embodiments where the stimulator 106 is connected to the implantable device 10, the voltage is applied across the stimulator 106 by the implantable device 10. For embodiments where the stimulator 106 is connected to the communication device 102, the voltage is applied across the stimulator 106 by the communication device 102.

From block 172, the process advances to block 174, where the analysis module 20 determines whether myocardial pH values and/or the left ventricular wall tension values measured after the start of the application of the voltage across the stimulator 106 are indicative of myocardial ischemia. From block 174, the process returns to block 172 or advances to block 176. If the determination made at block 174 is a determination that the myocardial pH values and/or the left ventricular wall tension values measured after the start of the application of the voltage across the stimulator 106 are indicative of myocardial ischemia, the process returns to block 172, where the process advances as described above. The process described for blocks 172-174 may be repeated any number of times. In general, the application of the voltage will continue as long as the measured myocardial pH values and/or the measured left ventricular wall tension values are indicative of myocardial ischemia.

If the determination made at block 174 is a determination that the myocardial pH values and/or the left ventricular wall tension values measured after the start of the application of the voltage across the stimulator 106 are not indicative of myocardial ischemia, the process advances from block 174 to block 176. At block 176, the voltage being applied across the stimulator 106 is disconnected. From block 176, the process returns to block 162, where the process advances as described above.

Nothing in the above description is meant to limit the invention to any specific materials, geometry, or orientation of elements. Many part/orientation substitutions are contemplated within the scope of the invention and will be apparent to those skilled in the art. The embodiments described herein were presented by way of example only and should not be used to limit the scope of the invention.

Although the invention has been described in terms of particular embodiments in this application, one of ordinary skill in the art, in light of the teachings herein, can generate additional embodiments and modifications without departing from the spirit of, or exceeding the scope of, the claimed invention. For example, many of the steps of the method 90 may be performed concurrently. Accordingly, it is understood that the drawings and the descriptions herein are proffered only to facilitate comprehension of the invention and should not be construed to limit the scope thereof.

What is claimed is:

1. An implantable device, wherein the implantable device comprises:
a computing device;
a microelectromechanical system (MEMS) pH sensor connected to the computing device; and
a communication system connected to the computing device.

2. The implantable device of claim 1, wherein the computing device is configured for applying a voltage across a stimulator connected to the implantable device.

3. The implantable device of claim 1, wherein the MEMS pH sensor comprises:
a first electrode;
a second electrode formed on the first electrode;
a first dielectric layer formed on the first electrode;
a third electrode formed on the first dielectric layer;
a second dielectric layer formed on the third electrode;
an electrolyte layer formed on the third electrode; and
a liquid junction connected to the second dielectric layer.

4. The implantable device of claim 3, wherein the MEMS pH sensor further comprises a passivation layer formed on the second dielectric layer.

5. The implantable device of claim 1, wherein the MEMS pH sensor comprises a microfluidic switch.

6. The implantable device of claim 1, wherein the MEMS pH sensor comprises:
a substrate;
a first electrode formed on the substrate;
a second electrode formed on the substrate;
a plurality of third electrodes formed on the substrate;
a cover connected to the substrate, wherein the cover defines a closed-loop fluidic channel between the substrate and a surface of the cover; and
a liquid junction connected to the cover.

7. The implantable device of claim 1, wherein the communication system comprises at least one of the following:
a transmitter;
a receiver; and
a transceiver.

8. The implantable device of claim 1, wherein the communication system comprises an antenna, wherein the antenna comprises:
a first pole, wherein the first pole comprises:
a first conductive layer; and
a first insulating layer connected to the first conductive layer; and
a second pole, wherein the second pole comprises:
a second conductive layer; and
a second insulating layer connected to the second conductive layer.

9. The implantable device of claim 1, further comprising a microelectromechanical system (MEMS) pressure sensor connected to the computing device.

10. The implantable device of claim 9, wherein the MEMS pressure sensor comprises a piezoresistive sensing member.

11. The implantable device of claim 1, further comprising an analysis module configured for determining the existence of organ ischemia, wherein the determination is based on one or more pH values measured by the MEMS pH sensor.

12. The implantable device of claim 11, wherein the determination is further based on one or more left ventricular wall tension values measured by a MEMS pressure sensor.

13. The implantable device of claim 1, further comprising a power source connected to the computing device.

14. The implantable device of claim 13, wherein the power source is a battery.

15. A system, comprising:
an implantable device, wherein the implantable device comprises:
a computing device;
a microelectromechanical system (MEMS) pH sensor connected to the computing device; and
a communication system connected to the computing device; and
a communication device connected to the implantable device.

16. The system of claim 15, wherein the implantable device further comprises a microelectromechanical system (MEMS) pressure sensor connected to the computing device.

17. The system of claim 15, wherein the communication device is wirelessly connected to the implantable device.

18. The system of claim 15, wherein the communication device is configured for communication with at least one device other than the implantable device.

19. The system of claim 15, further comprising a power source operatively connected to the implantable device.

20. The system of claim 19, wherein the power source is a piezoelectric energy harvesting device.

21. The system of claim 19, wherein the power source is a biofuel cell.

22. The system of claim 19, wherein the power source is a volume conduction energy delivery device.

23. The system of claim 15, further comprising a stimulator connected to the implantable device.

24. A method, implemented at least in part with a computing device, the method comprising:
measuring pH values of an organ with an implanted device; and
determining whether organ ischemia exists based on at least one of the measured pH values.

25. The method of claim 24, wherein determining whether organ ischemia exists comprises determining whether the at least one of the measured pH values is less than a threshold value.

26. The method of claim 24, further comprising sending an alert signal when it is determined that organ ischemia exists.

27. The method of claim 24, further comprising measuring left ventricular wall tension values with the implanted device.

28. The method of claim 27, wherein determining whether organ ischemia exists further comprises determining based on at least one of the measured left ventricular wall tension values.

29. The method of claim 28, wherein determining whether organ ischemia exists further comprises determining whether the at least one of the measured left ventricular wall tension values is less than a threshold value.

30. The method of claim 24, further comprising stimulating at least one cardiac vagal nerve branch when it is determined that organ ischemia exists.

31. The method of claim 30, wherein stimulating the at least one cardiac vagal nerve branch comprises the implantable device applying a voltage across a stimulator connected to the implantable device and the at least one cardiac vagal nerve stimulator.
